(19) United States
(12) Patent Application Publication
(10) Pub. No.: US 2023/0400395 A1
(43) Pub. Date: Dec. 14, 2023

(54) PREPARATION OF LIPID NANOPARTICLE ENCAPSULATED NUCLEIC ACID SAMPLES FOR ACCURATE COMPOSITIONAL ANALYSIS

(71) Applicant: Waters Technologies Corporation, Milford, MA (US)

(72) Inventors: Matthew A. Lauber, North Smithfield, RI (US); Bonnie Alden, Whitinsville, MA (US); Mame Maissa Gaye, Cambridge, MA (US)

(73) Assignee: Waters Technologies Corporation, Milford, MA (US)

(21) Appl. No.: 18/328,722

(22) Filed: Jun. 3, 2023

Related U.S. Application Data
(60) Provisional application No. 63/414,131, filed on Oct. 7, 2022.
(60) Provisional application No. 63/350,059, filed on Jun. 8, 2022.

(51) Int. Cl. G01N 1/40; C12Q 1/6806

(52) U.S. Cl. CPC G01N 1/4055; C12Q 1/6806

(57) ABSTRACT

The present disclosure is directed to methods of sample preparation from lipid nanoparticle (LNP)-encapsulated nucleic acids utilizing solid-phase extraction techniques.

### CROSS-REFERENCE TO RELATED APPLICATIONS

This patent application claims priority to and the benefit of U.S. Provisional Application No. 63/414,131, filed Oct. 7, 2022. This application also claims priority to and the benefit of U.S. Provisional Application No. 63/350,059, filed Jun. 8, 2022. Each of the foregoing applications is herein incorporated by reference in its entirety.

### TECHNICAL FIELD

The present disclosure relates to methods of sample preparation from lipid nanoparticle (LNP)-encapsulated nucleic acids utilizing solid-phase extraction techniques. The methods utilize sorbents with an anion-exchange functionality to separate the nucleic acid from the various lipid components present in such samples, enabling secondary analysis of the nucleic acid, the lipid components, or both.

### BACKGROUND

Lipid nanoparticle (LNP)-encapsulated nucleic acids have found utility as therapeutic agents, e.g., for modulating protein expression or as novel vaccine platforms. For example, the current COVID-19 messenger RNA (mRNA) vaccines from Pfizer-BioNTech and Moderna utilize LNPs to encapsulate mRNA, shield it from destructive enzymes, and shuttle it into cells. The LNPs used in these COVID-19 vaccines contain 1) ionizable lipids, Whose positive charges bind to the negatively charged phosphate backbone of mRNA; 2) polyethylene glycol linked (PEGylated) lipids that help stabilize the nanoparticle; and 3) phospholipids and cholesterol molecules that contribute to the nanoparticle's structure. Thousands of these lipid components surround the mRNA. Further, various lipid molecules, both naturally occurring and synthetic, are being evaluated for their LNP structure-function and stability properties. These evaluations require optimization of stoichiometry. For analysis of both existing LNP-based nucleic acid drug products and developmental research, analytical methods for quantifying the lipid components and/or nucleic acid present in such LNP-based nucleic acid products are needed.

Accordingly, it would be desirable in the art to provide sample preparation techniques for LNP-encapsulated nucleic acids as well as analytical approaches to facilitate the separation and quantification of the lipid components present, the nucleic acid present, or both. It is further desirable to provide stability/potency indicating analyses of the nucleic acid drug substance present in such LNP-encapsulated nucleic acids.

### SUMMARY

The present technology is generally directed to methods for preparing a sample from a lipid nanoparticle (LNP)-encapsulated nucleic acid. The methods generally feature use of a combination of dissolution and solid phase extraction techniques to separate the lipid components from the nucleic acid present in the LNP-encapsulated nucleic acid. In particular, methods of the present technology feature dissolving a sample of an LNP-encapsulated nucleic acid in an organic solvent to provide a solution comprising dissolved lipids and solubilized nucleic acid, and passing the solution through a weak anion exchange (WAX) solid phase sorbent. The lipid components pass through the sorbent to form a lipid sample which may be further analyzed to determine the composition, stoichiometry, and the like. Optionally, the nucleic acid may be eluted from the sorbent to form a nucleic acid sample which may be further analyzed, subject to additional processing, or both. Surprisingly, it has been found according to the present disclosure that use of certain organic solvents sufficiently disrupts intermolecular interactions between the lipid components and the nucleic acid, while the WAX sorbent effectively adsorbs the nucleic acid, providing a lipid sample with high recovery, without altering the relative abundances of the various lipids present in the initial LNP-encapsulated nucleic acid, and which is substantially free of nucleic acid and ready for further analysis. Further surprising is that the nucleic acid can be obtained with good recovery by eluting from the WAX sorbent with a pH-adjusted eluant (e.g., an aqueous buffer). Advantageously, the nucleic acid sample thus prepared is substantially free of lipid components and is in a form suitable for direct further analysis. In other embodiments, the nucleic acid retained on the sorbent may be subjected to nuclease digestion while still on sorbent, and the resulting digested nucleic acid components eluted and subsequently analyzed. The disclosed methods are operationally simple and rapid, and enable accurate analyses of all components present in, e.g., a LNP-encapsulated nucleic acid drug product. Such analyses are useful in determining or confirming composition, stoichiometry, purity, stability, and the like of such drug products, and may also find utility in the process of developing new lipid nanoparticle-based therapeutic agents and vaccines.

Accordingly, in one aspect, the present technology is directed to a method of preparing a sample for compositional analysis from a lipid nanoparticle encapsulated nucleic acid, the method comprising:
dissolving a sample comprising the lipid particle encapsulated nucleic acid in an organic solvent to provide a solution comprising dissolved lipids and solubilized nucleic acid; and
adsorbing the solubilized nucleic acid on a weak anion exchange (WAX) solid phase sorbent.

In some embodiments, the sample comprising the lipid particle encapsulated nucleic acid is a sample of a formulated LNP-encapsulated nucleic acid drug product.

In some embodiments, the WAX solid phase sorbent comprises a surface having anion exchanger residues disposed thereon, the anion exchanger residues having a pKa in a range from about 5 to about 11. In some embodiments, the anion exchanger residues have a pKa in a range from about 8 to about 10.

In some embodiments, the anion exchanger residues comprise a primary amine, a secondary amine, a tertiary amine, or a combination thereof. In some embodiments, the anion exchanger residues comprise piperazine.

In some embodiments, the organic solvent comprises a C1-C4 alcohol, acetonitrile, dimethyl formamide, dimethyl sulfoxide, or a combination thereof. In some embodiments, the organic solvent is methanol. In some embodiments, the organic solvent is n-propanol.

In some embodiments, the organic solvent further comprises an additive selected from the group consisting of acids, bases, detergents, buffers, salts, and combinations thereof. In some embodiments, the additive comprises an ionizable cationic base. In some embodiments, the ionizable cationic base comprises ammonium or trialkylammonium ions.

In some embodiments, the method further comprises removing the dissolved lipids from the WAX solid phase sorbent, forming a lipid sample which is substantially free of nucleic acid. In some embodiments, removing the dissolved lipids comprises allowing the dissolved lipids to pass through the WAX solid phase sorbent. In some embodiments, removing the dissolved lipids further comprises flowing the organic solvent through the WAX solid phase sorbent and collecting the organic solvent flowed therethrough to provide one or more washings; and combining the one or more washings with the dissolved lipids which have been allowed to pass through the WAX solid phase sorbent.

In some embodiments, method further comprises analyzing the lipid sample by high performance liquid chromatography coupled with a suitable detection method, such as tunable, dual wavelength ultraviolet/visible (TUV) detection, evaporative light scattering, mass spectrometry, charged aerosol, or refractive index.

In some embodiments, method further comprises eluting the adsorbed nucleic acid from the WAX solid phase sorbent, forming a nucleic acid sample which is substantially free of lipids. In some embodiments, eluting comprises flowing an aqueous buffer through the WAX solid phase sorbent, wherein the aqueous buffer has a pH greater than or equal to the pKa of the anion exchanger residues of the WAX solid phase sorbent.

In some embodiments, the aqueous buffer comprises up to about 50% by volume of a non-aqueous solvent. In some embodiments, the non-aqueous solvent comprises a C1-C4 alcohol, acetonitrile, dimethyl formamide, dimethyl sulfoxide, or a combination thereof.

In some embodiments, the aqueous buffer has a pH of about 8 to about 12.5, or from about 8.5 to about 12. In some embodiments, the aqueous buffer is an ammonium bicarbonate solution having a pH of about 11.

In some embodiments, eluting further comprises flowing an organic solvent through the WAX solid phase sorbent and collecting the organic solvent flowed therethrough to provide one or more washings; and combining the one or more washings with the nucleic acid which has been eluted from the WAX solid phase sorbent with the aqueous buffer.

In some embodiments, the method further comprises analyzing the nucleic acid sample by high performance liquid chromatography coupled with a suitable detection method, such as tunable, dual wavelength ultraviolet/visible (TUV) detection, evaporative light scattering, mass spectrometry, charged aerosol, or refractive index.

In some embodiments, the method further comprises digesting at least a portion of the nucleic acid present in the nucleic acid sample with a nuclease.

In some embodiments, the method further comprises hydrolyzing at least a portion of the nucleic acid present in the nucleic acid to form a mixture of constituent residues.

In some embodiments, the method comprises digesting at least a portion of the solubilized nucleic acid adsorbed on the sorbent, forming an adsorbed mixture of nucleic acid digestion products, wherein digesting comprises contacting the sorbent with a nuclease; and eluting the mixture of nucleic acid digestion products from the WAX solid phase sorbent, forming a nucleic acid digestion sample.

In some embodiments, eluting comprises flowing an aqueous buffer through the WAX solid phase sorbent, wherein the aqueous buffer has a pH greater than or equal to the pKa of the anion exchanger residues of the WAX solid phase sorbent, and wherein said aqueous buffer comprises up to about 50% by volume of a non-aqueous solvent.

In some embodiments, the non-aqueous solvent comprises a C1-C4 alcohol, acetonitrile, dimethyl formamide, dimethyl sulfoxide, or a combination thereof.

In some embodiments, the aqueous buffer has a pH of about 7 to about 12.5, or from about 8 to about 12.

In some embodiments, the method further comprises analyzing the nucleic acid digestion sample by high performance liquid chromatography coupled with a suitable detection method, such as tunable, dual wavelength ultraviolet/visible (TUV) detection, evaporative light scattering, mass spectrometry, charged aerosol, or refractive index.

### BRIEF DESCRIPTION OF THE DRAWINGS

In order to provide an understanding of embodiments of the technology, reference is made to the appended drawings, which are not necessarily drawn to scale. The drawings are exemplary only and should not be construed as limiting the technology. The disclosure described herein is illustrated by way of example and not by way of limitation in the accompanying figures.

FIG. 1 is a schematic illustration of a method for preparing a lipid sample according to a non-limiting embodiment of the disclosure.

FIG. 2 is a further schematic illustration corresponding to the method illustrated in FIG. 1, and providing the chemical principles believed to be associated with the method.

FIG. 3 is a schematic illustration of further methods for preparing a sample according to additional non-limiting embodiments of the disclosure.

FIG. 4 is a schematic illustration of a method for preparing a nucleic acid digestion sample according to additional non-limiting embodiments of the disclosure.

FIGS. 5A and 5B depict exemplary chromatographic separations of lipid components in a sample according to a non-limiting embodiment of the disclosure.

FIGS. 6A, 6B and 6C depict exemplary chromatographic separations of a nucleic acid in a sample according to a non-limiting embodiment of the disclosure.

FIG. 7A depicts an exemplary chromatographic separation of an mRNA sample prepared in pH 7.5 buffer.

FIG. 7B depicts an exemplary chromatographic separation of a sample from solution phase nuclease digestion of the mRNA sample of FIG. 7A.

FIG. 7C depicts an exemplary chromatographic separation of a lipid-encapsulated mRNA sample subjected to solution phase nucleic acid digestion in a disrupting buffer solution.

FIG. 8A depicts an exemplary chromatographic separation of a nucleic acid digestion sample prepared according to additional non-limiting embodiments of the disclosure.

FIG. 8B depicts an exemplary chromatographic separation of a nucleic acid digestion sample prepared by nucleic acid digestion on sorbent of a lipid-encapsulated mRNA sample prepared according to an additional non-limiting embodiment of the disclosure.

FIGS. 9A and 9B are expanded views of the exemplary chromatographic separations of FIGS. 8A and 8B, respectively.

### DETAILED DESCRIPTION

Before describing several example embodiments of the technology, it is to be understood that the technology is not limited to the details of construction or process steps set forth in the following description. The technology is capable of other embodiments and of being practiced or being carried out in various ways.

### Definitions

With respect to the terms used in this disclosure, the following definitions are provided. This application will use the following terms as defined below unless the context of the text in which the term appears requires a different meaning

The articles “a” and “an” are used herein to refer to one or to more than one (i.e., to at least one) of the grammatical object of the article.

The term “about” used throughout this specification is used to describe and account for small fluctuations. For example, the term “about” can refer to less than or equal to ±5%, such as less than or equal to ±2%, less than or equal to ±1%, less than or equal to ±0.5%, less than or equal to ±0.2%, less than or equal to ±0.1% or less than or equal to ±0.05%. All numeric values herein are modified by the term “about,” whether or not explicitly indicated. A value modified by the term “about” of course includes the specific value. For instance, “about 5.0” must include 5.0.

As used herein, the term “substantially free” (e.g., with reference to a nucleic acid or lipid component) means that the sample contains an amount below a level which would create interference in a subsequent assay, or an undetectable amount. This amount may vary depending on the specific component and the assay to which it is subjected, but is generally less than about 1% by weight, or less than 0.5% by weight, or less than 0.1% by weight, or less than 0.01% by weight, or less than 0.001% by weight, or even 0% by weight of the relevant component.

The term “lipid” as used herein refers to a diverse group of organic compounds including fats, oils, and certain components of biological membranes which are characterized by a lack of appreciable interaction with water (i.e., exhibiting hydrophobicity). Lipids encompass molecules such as fatty acids and their derivatives (including tri-, di-, monoglycerides, and phospholipids), as well as other sterol-containing metabolites (e.g., cholesterol). Lipid molecules which may be present in a lipid nanoparticle-encapsulated nucleic acid sample are further described herein below.

The term “nucleic acid” as used herein refers to linear biopolymer chains composed of series of nucleotides, which may also be referred to as “polynucleotides.” The term “nucleotide” as used herein refers to monomeric units consisting of three components: a 5-carbon sugar, a phosphate group, and a nitrogenous base. The chain length (number of nucleotides) of a nucleic acid may vary depending on, for example, the source and intended use. For example, a nucleic acid may comprise a relatively short chain (e.g., on the order of about 2 to about 20, or about 10 to about 200 nucleotides, generally referred to as oligonucleotides), or may comprise much larger chains (e.g., thousands, millions, or more nucleotides).

The two main classes of nucleic acids are ribonucleic acid (RNA) and deoxyribonucleic acid (DNA). If the 5-carbon sugar is D-ribose, the polymer is RNA, which is formed from chains of nucleotides joined by phosphodiester bonds between the 3′-hydroxyl group of one ribose unit and the 5′-hydroxyl group of an adjacent ribose unit. The nucleotide nitrogenous bases of RNA are adenine, guanine, cytosine, and uracil. If the 5-carbon sugar is 2-deoxyribose, the polymer is DNA, which is formed from chains of nucleotides joined by phosphodiester bonds between the 3′-hydroxyl group of one 2-deoxyribose unit and the 5′-hydroxyl group of an adjacent 2-deoxyribose unit. The nucleotide nitrogenous bases of DNA are adenine, guanine, cytosine, and thymine.

Generally, DNA acts as a store for encoded genetic information for a cell or organism. A segment of DNA that codes for a cell's synthesis of a specific protein is a gene. Within eukaryotic cells, DNA is organized into dense protein-DNA complexes (chromosomes). Generally, RNA, in the form of messenger, ribosomal, or transfer RNA, copies the genetic message from DNA and serves various roles in forming peptides encoded by the DNA.

Reference to a nucleic acid herein contemplates any nucleotide chain including, but not limited to, RNA, DNA, oligonucleotides, aptamers, and analogs or derivatives of any thereof, such as nucleotides comprising modified (i.e., artificial) bases or sugars. Further, reference to RNA and DNA include all forms thereof, such as may be present in a genome, chromosome, histone, or an isolated gene, those present either naturally or as artificially introduced into cells or viruses, those encoding genetic information or peptide sequences, and artificial, truncated, and/or fragments versions of any of the foregoing.

Embodiments of the present disclosure are now described in detail with the understanding that such embodiments are exemplary only. Such embodiments constitute what the inventors now believe to be the best mode of practicing the technology. Those skilled in the art will recognize that such embodiments are capable of modification and alteration.

Synthetic nucleic acids are providing a new vaccine format and represent a new means of modulating protein expression. Whether based on mRNA or chemically synthesized and purified small oligonucleotides, therapeutics comprising nucleic acids are demonstrating great potential in the realm of healthcare. A major breakthrough that has helped unlock the potential of nucleic acids as therapeutics has been the formulation of lipid components as lipid nanoparticles (LNP) to effectively encapsulate and thereby provide a vehicle for cellular endocytosis of nucleic acids such as mRNA. Cationic lipids are at the heart of this technology. They serve as ion pairs with and counter ions to the negatively charged (phosphate) backbones of the nucleic acid molecules which serve to stabilize the construct through these intermolecular interactions. The nucleic acids adopt structures that can be further built upon with cholesterol and zwitterionic phosphatidyl choline lipids, and ultimately with amphipathic hydrophilic counterparts, to yield solid lipid nanoparticles comprising the nucleic acid. Examples of recent drug products include ONPATTRO™ (Patisiran; Alynylam), a lipid nanoparticle-based short interfering RNA (siRNA) drug for the treatment of polyneuropathies induced by hereditary transthyretin amyloidosis, and the Pfizer-BioNTech and Moderna COVID-19 RNA vaccines.

Various lipids, both natural occurring and synthetic, are being explored to identify advantageous structure-function and stability properties for application in further LNP-based therapeutic agents. It is important to have analytical methods which accurately indicate the stoichiometry of the lipid and/or nucleic acid components present in newly designed constructs, and to establish stability and purity of available vaccines and therapeutics.

Presently, a preferred method of measuring the lipid composition of an LNP-encapsulated nucleic acid drug comprises dissolving the LNP-encapsulated nucleic acid particle in an organic solvent and performing reversed phase chromatography for separation, coupled with detection and quantitation by e.g., tunable, dual wavelength ultraviolet/visible (TUV) detection, evaporative light scattering, charged aerosol, or mass spectrometric means. In such analyses, the disruption of lipid-nucleic acid interactions is paramount. The quantitation of each lipid component is dependent on their separation into individually chromatographic bands. If strong intermolecular interactions between sample components remain undisrupted, the quantitation of a lipid component, e.g., the cationic lipid component, can be negatively influenced, resulting in under reporting and an inaccurate analysis.

Similar complications can occur when the analyte of interest is the nucleic acid component of a LNP-encapsulated nucleic acid drug product. For example, if cationic lipids remain bound to the nucleic acid, detection of the nucleic acid will be confounded by its splitting into one or more additional chromatographic bands, corresponding to different extents of non-covalently bound cationic lipid. In another example, the incomplete displacement of lipids from the nucleic acid can potentially compromise nuclease digestion, which is used to facilitate secondary analysis of the nucleic acid drug substance.

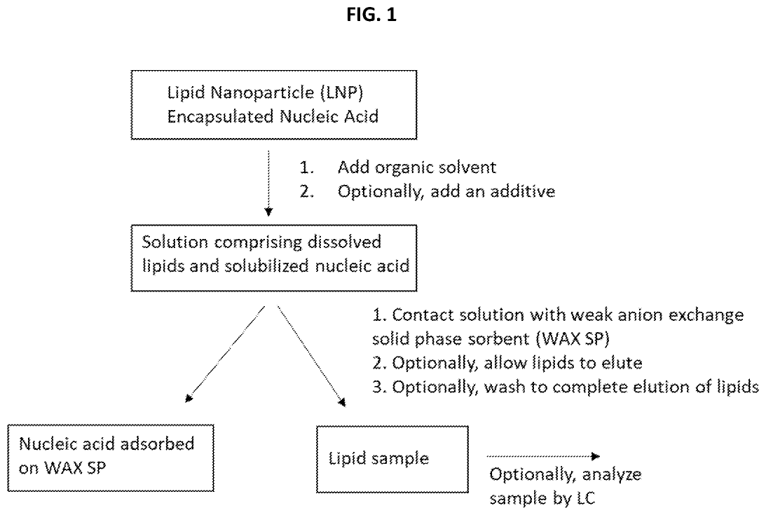

In view of these challenges, it would be desirable to provide sample preparation methods which assure the complete disruption of lipid-nucleic acid interactions, providing lipid component and/or nucleic acid samples ready for further analysis. Accordingly, the present disclosure provides a method of sample preparation which utilizes sorbents having an anion-exchange functionality. The method effectively and readily separates the nucleic acid from the various lipid components, enabling a variety of secondary analyses of the nucleic acid, the lipid components, or both. The method generally includes dissolving a lipid encapsulated nucleic acid through the addition of an organic solvent and partitioning the lipids away from the nucleic acid through use of anion exchange solid phase extraction. A flowchart depicting the general method according to one non-limiting embodiment of the disclosure is provided in FIG. 1. Each of the method steps, and methods of performing subsequent separation and/or quantitation of the lipid components, the nucleic acid, or both, are described further herein below.

With reference to FIG. 1, a lipid nanoparticle (LNP)-encapsulated nucleic acid is provided. In some embodiments, the lipid nanoparticle (LNP)-encapsulated nucleic acid is a drug product or vaccine. In some embodiments, the LNP-encapsulated nucleic acid is a vaccine against a viral infection, such as COVID-19. The LNP-encapsulated nucleic acid comprises a nucleic acid, which may be a deoxyribonucleic acid (DNA), a ribonucleic acid (RNA), or an analog of either thereof (e.g., a synthetic variant, including, but not limited to, antisense oligonucleotides (ASOs)).

In some embodiments, the nucleic acid is a DNA. In some embodiments, the DNA is single-stranded DNA. In some embodiments, the DNA is double-stranded DNA.

In some embodiments, the nucleic acid is an RNA. In some embodiments, the RNA is single-stranded RNA. In some embodiments, the DNA is double-stranded RNA. In some embodiments, the nucleic acid is a messenger RNA (mRNA), which is a single-stranded RNA that corresponds to the genetic sequence of a gene used a template during protein synthesis. Recently, mRNA has become an important therapeutic agent for the treatment of viruses and genetic diseases. In some embodiments, the nucleic acid is a single guide RNA (sgRNA), a small interfering RNA (siRNA), a small hairpin RNs (shRNA), a transfer RNA (tRNA), or a micro-RNA (miRNA).

In some embodiments, the nucleic acid is a plasmid, which is a small, circular nucleic acid molecule found in certain microscopic organisms (e.g., bacteria), and which finds utility in, for example, genetic engineering and gene therapy techniques. Plasmids may be double-stranded DNA, single-stranded DNA, or double-stranded RNA.

The LNP-encapsulated nucleic acid comprises various lipids. For example, an LNP-encapsulated nucleic acid sample as disclosed herein typically includes two, three, four, or more different types of lipids, such as one or more phospholipids, one or more PEGylated lipids, one or more ionizable lipids, cholesterol, or a combination thereof. Each of these lipids serves a particular function in creating and stabilizing the lipid nanoparticle. Particularly, cationic lipids have a positively charged ammonium group which associates with a negatively charged phosphate group in the nucleic acid backbone.

In some embodiments, the LNP-encapsulated nucleic acid comprises a phospholipid. The term “phospholipid” as used herein refers to an amphiphilic lipid having a negatively charged phosphate group and a hydrophobic moiety. Phospholipids are made up of two fatty acid tails (hydrophobic moiety) and a phosphate group head, connected via glycerol, and may be natural or synthetic. Non-limiting examples of phospholipids include soybean lecithin, egg lecithin, phosphatidylglycerols, phosphatidylinositols, phosphatidylethanolamines, phosphatidic acids, sphingomyelin, diphosphatidylglycerols, phosphatidylserine, phosphatidylcholines, dimyristoylphosphatidylcholine, dimyristoylphosphatidylglycerol, distearoylphosphatidylglycerol, dipalmitoylphosphatidylcholine, and hydrogenated or partially hydrogenated lecithins, In some embodiments, the phospholipid is a phosphatidic acid (DMPA, DPPA, DSPA), a phosphatidylcholine (DDPC, DLPC, DMPC, DPPC, DSPC, DOPC, POPC, DEPC), a phosphatidylglycerol (DMPG, DPPG, DSPG, POPG), a phosphatidylethanolamine (DMPE, DPPE, DSPE DOPE), or a phosphatidylserine (DOPS). In some embodiments, the phospholipid is 1,2-distearoyl-sn-glycero-3-phosphocholine (1,2-DSPC).

In some embodiments, the LNP-encapsulated nucleic acid comprises a polyethylene glycol (PEG)-modified lipid, also referred to herein as a “pegylated lipid”, “PEGylated lipid” or a “PEG-ylated lipid”; all three terms can be used interchangeably. The term “pegylated lipid” as used herein refers to a lipid having a polyethylene glycol strand covalently bound thereto. Reference to a pegylated lipid includes pegylated phospholipids and other lipid categories. In some embodiments, the pegylated lipid is 2-[(polyethylene glycol)-2000]-N,N-ditetradecylacetamide (ALC-0159).

In some embodiments, the LNP-encapsulated nucleic acid comprises an ionizable (e.g., cationic) lipid. The term “ionizable lipid” as used herein refers to a lipid having an amino group which may be protonated under conditions of an acidic pH to yield an ammonium cation but remain neutral with respect to charge at physiological pH. Examples of ionizable lipids include, but are not limited to, 4-(dimethylamino)-butanoic acid, (10Z,13Z)-1-(9Z,12Z)-9,12-octadec adien-1-yl-10,13-nonadecadien-1-yl ester (MC3), 8-[(2-hydroxyethyl)[6-oxo-6-(undecyloxy)hexyl] amino]-octanoic acid, 1-octylnonyl ester (SM-102), and ([(4-hydroxybutyl)azanediyl]di(hexane-6,1-diyl) bis(2-hexyldecanoate (ALC-0315). In some embodiments, the ionizable lipid is ([(4-hydroxybutyl)azanediyl]di(hexane-6,1-diyl) bis(2-hexyldecanoate (ALC-0315).

In some embodiments, the LNP-encapsulated nucleic acid comprises a sterol, such as cholesterol.

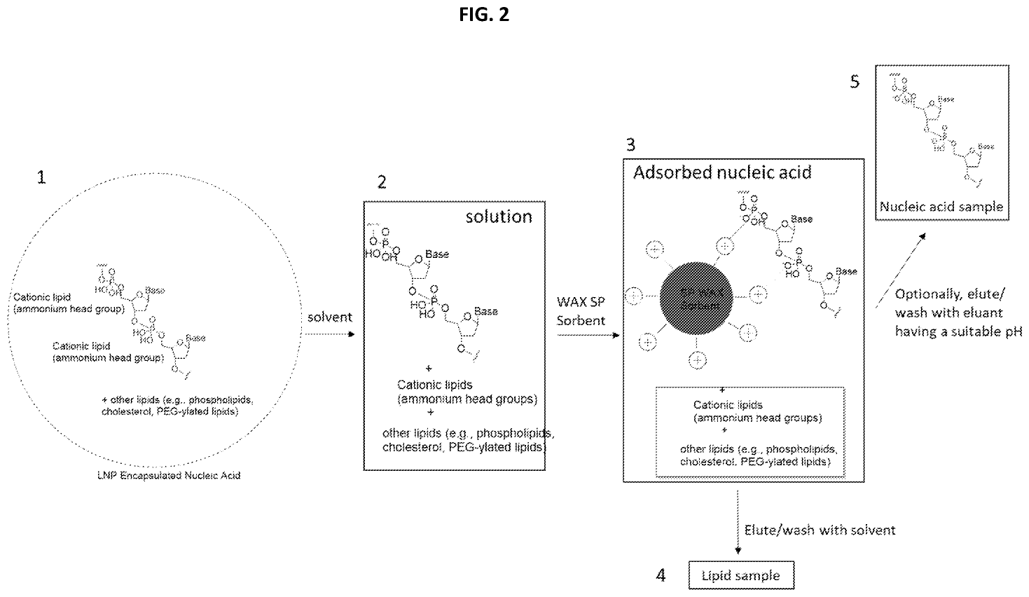

The disclosed method comprises dissolving the sample in an organic solvent to provide a solution comprising dissolved lipids and solubilized nucleic acid. With continued reference to FIG. 1., the LNP-encapsulated nucleic acid is treated with an organic solvent to dissolve the LNP-encapsulated nucleic acid, forming a solution comprising the solubilized nucleic acid and dissolved lipids. As used herein, the term “organic solvent” refers to any carbon-based molecule capable of dissolving or dispersing one or more other substances (e.g., one or more components of the LNP-encapsulated nucleic acid). Suitable solvents are generally polar and may be protic or aprotic. Suitable organic solvents include, but are not limited to, alcohols, nitriles, amides, sulfoxides, and the like. In some embodiments, the organic solvent comprises an alcohol having from 1 to 4 carbons (C1-C4 alcohol), acetonitrile, dimethyl formamide, dimethyl sulfoxide, or a combination thereof. Examples of C1-C4 alcohols include methanol, ethanol, n-propanol, isopropanol, n-butanol, isobutanol, and sec-butanol. In particular embodiments, the organic solvent is n-propanol. The addition of the organic solvent serves to at least partially disrupt interactions between cationic lipid molecules and the nucleic acid, and dissolves the remaining lipid components (e.g., cholesterol, phospholipids, PEG-ylated lipids). A cartoon illustration of the dissolution process is provided in FIG. 2. With reference to FIG. 2, the LNP-encapsulated nucleic acid 1 is treated with the organic solvent to provide a solution 2 comprising the nucleic acid, cationic lipids, and other lipids (e.g., cholesterol, phospholipids, PEG-ylated lipids).

In some embodiments, the method further comprises utilizing an additive in the organic solvent to facilitate disruption of the intermolecular bonds holding the LNP particle together. Suitable additives include, but are not limited to, detergents, buffers, salts, acids, bases (including ionizable cationic bases), and combinations thereof. In some embodiments, the additive comprises or is ammonium hydroxide, acetic or formic acid, triethylamine, Tris, HEPES, a phosphate, Bis-Tris, tetramethylammonium, hexylamine, diisopropylethylamine, a salt of a trialkylamine, ammonium bicarbonate, ammonium acetate, ammonium formate, or a combination thereof. In some embodiments, the additive is present in the organic solvent in the form of an aqueous solution. In some embodiments, the aqueous solution, the organic solution further comprising the additive, or both, have a pH value in a range of about 6 to about 12, such as from about 6, about 7, about 8, or about 9, to about 10, about 11, or about 12. In some embodiments, the aqueous solution, the organic solvent further comprising the aqueous solution, or both, have a pH in a range from about 6 to about 9. In some embodiments, the organic solvent further comprises triethylammonium acetate. In some embodiments, the organic solvent is a mixture of n-propanol and 0.1M triethylammonium acetate (pH 10 buffer).

The method comprises adsorbing the solubilized nucleic acid on a weak anion exchange (WAX) solid phase extraction (SPE) sorbent. With continued reference to FIG. 1., the obtained solution is loaded onto a WAX SPE sorbent bed. The lipids are allowed to flow through (elute) and are to form a lipid sample, while the nucleic acid is left adsorbed to the sorbent. Without wishing to be bound by theory, it is believed that the adsorption of nucleic acid onto WAX sorbent anion exchanger ligands serves to displace and purify away the cationic lipids. With further reference to FIG. 2, it is believed that cationic groups on the surface of the WAX sorbent associate with negatively charged phosphate groups in the nucleic acid, causing the nucleic acid to be adsorbed (retained) 3 on the sorbent. In contrast, the cationic and uncharged lipids are unretained by the sorbent, and either flow through, or are further washed through (eluted) with a solvent (e.g., an organic solvent as described herein, and which may be the same of different than the organic solvent used for the dissolution) to provide the lipid sample 4.

In some embodiments, the method further comprises analyzing the lipid sample, such as by a suitable liquid chromatographic method. For example, the lipid sample comprising the lipid components (i.e., unretained and/or eluted lipids) may be analyzed to measure their relative abundance, to investigate impurities which may be present in the sample, or both. Suitable liquid chromatographic methods include high performance and ultra-high performance liquid chromatography coupled with various detection methods including, but not limited to, tunable, dual wavelength ultraviolet/visible (TUV) detection, evaporative light scattering, mass spectrometry, charged aerosol, or refractive index.

The WAX sorbent used may vary. Generally, suitable sorbents include those having weakly basic to strongly basic anion exchanger residues disposed on the sorbent surface. Examples of suitable anion exchanger residues include, but are not limited to, primary, secondary, and tertiary amines, and combination thereof. In some embodiments, the anion exchanger residues comprise tertiary amino groups. In some embodiments, the anion exchanger residues have a pKa in a range from about 5 to about 11, such as from about 5, about 6, about 7, or about 8, to about 9, about 10, or about 11. In some embodiments, the anion exchanger residues have a pKa in a range from about 8 to about 10. Without wishing to be bound by theory, it is believed that selection of sorbents having anion exchanger residues with particular pKa ranges and/or degrees of hydrophobicity may provide enhanced recovery of nucleic acids when matched to the basicity and hydrophobicity of the nucleic acid. In some embodiments, the WAX sorbent comprises polyethyleneimine, diethylaminoethyl, diethylaminopropyl, or dimethylaminopropyl residues. In some embodiments, the WAX sorbent comprises piperazine residues.

The WAX sorbent comprises sorbent particles. The composition of the sorbent particles may vary. For example, suitable sorbent materials include silica, organosilica, and polymeric particles, which comprise or are modified (e.g., by bonding or grafting) to comprise anionic residues as described herein.

The particle size of the sorbent may vary. In some embodiments, the sorbent has a particle size in a range from about 10 to about 100 μm in diameter, such as from about 20 to 70 μm in diameter.

The solid phase sorbent is typically a porous material. The pore size of the sorbent may vary. In some embodiments, the sorbent has an average pore diameter in a range from about 50 to about 3000 Å. In some embodiments, the sorbent has an average pore diameter in a range from about 50 to about 100 Å. Without wishing to be bound by theory, it is believed that selection of pore diameter may influence adsorption and elution behavior depending on the specific nucleic acid present. For example, in some embodiments, multimodal retention mechanisms may take place between the sorbent and the nucleic acid. Specifically, larger pore diameters may allow for some nucleic acids, in part or whole, to adsorb within the intra-particle surface and experience significant amounts of restricted diffusion upon elution.

In some embodiments, the WAX sorbent comprises porous polymeric particles having an average pore size of about 80 μm diameter, modified with tertiary amino groups. One suitable example of such a WAX sorbent is the Oasis™ sorbent, available from Waters, Inc. (Milford, MA), in cartridge or multi-well plate format. In other embodiments, the sorbent is Sep-Pak™ Aminopropyl (Waters, Milford, MA), Poros 50 D (Dimethylaminopropyl, ThermoFisher Scientific, Waltham, MA), Poros 50 PI (Polyethyleneimine, ThermoFisher Scientific, Waltham, MA), UniDEAE-30S (Suzhou NanoMicro Technologies, China), UniBPC 55-WAX (Suzhou NanoMicro Technologies, China), UniDEAE-50XS (Suzhou NanoMicro Technologies, China), silica bonded with polyethyleneimine (WAX, Separation Methods Technologies, Newark, DE), silica bonded with diethylaminoethyl ligands (DEAE, Separation Methods Technologies, Newark, DE), or Clarity OTX (Phenomenex, Torrance, CA).

In some embodiments, the lipid components are obtained in the lipid sample with a recovery greater than about 80% by area, based on the total area of the lipid components present in the LNP-encapsulated nucleic acid (i.e., in the sample prior to performing the disclosed method). In some embodiments, the relative abundances of the lipids present are unchanged in the lipid sample relative to the abundances of the lipid components present in the LNP-encapsulated nucleic acid (i.e., in the sample prior to performing the disclosed method).

In some embodiments, the method further comprises eluting the adsorbed nucleic acid from the WAX solid phase sorbent, forming a nucleic acid sample which is substantially free of lipids. With continued reference to FIG. 2, generally, an eluant having a pH equal to or higher than the pKa of the cationic residues on the WAX sorbent is utilized to elute the adsorbed nucleic acid, providing the nucleic acid sample 5.

The eluant utilized for elution of the adsorbed nucleic acid may vary but is generally an aqueous buffer solution having a high pH (e.g., greater than about 7). In some embodiments, the eluant is an aqueous buffer having a pH in a range from about 8 to about 12.5, such as from about 8, or about 9, to about 10, about 11, or about 12. In some embodiments, the eluant is an aqueous buffer has a pH in a range from about 8.5 to about 12, such as about 8.5, about 9, about 9.5, about about 10.5, about 11, about 11.5, or about 12. Suitable buffers include, but are not limited to, aqueous ammonium bicarbonate, ammonium acetate, ammonium formate, triethylamine, tetramethylammonium, hexylamine, diisopropylethylamine, and ammonium hydroxide. In particular embodiments, the eluant is 100 mM ammonium bicarbonate buffer having a pH of about 11. In some embodiments, the eluant further comprises a non-aqueous solvent. The non-aqueous solvent may be present in various ratios with the aqueous buffer, and such ratios may be determined experimentally by one of skill in the art. In some embodiments, the eluant comprises up to about 50% by volume of the non-aqueous solvent. In some embodiments, the non-aqueous solvent is an organic solvent as described herein. Accordingly, in some embodiments, the eluant further comprises an organic solvent selected from the group consisting of methanol, ethanol, n-propanol, isopropanol, n-butanol, and acetonitrile.

Following the initial elution, one or more washes of the sorbent may be performed in order to enhance recovery of the nucleic acid. Accordingly, in some embodiments, the method further comprises flowing an organic solvent as described herein through the WAX solid phase sorbent and collecting the organic solvent flowed therethrough to provide one or more washings. In some embodiments, the one or more washings are combined with each other and/or with the initially eluted nucleic acid fraction. Such washes, when utilized, may be optimized based on the nature of the sorbent, the nucleic acid, or the sample as a whole. For example, the wash conditions may be optimized to accommodate sample hydrophobicity, matrix effects, lipid composition, or the presence of polymers and excipients in the sample. Optimization of the washes may comprise the addition of acids or bases, use of alternative protic or aprotic organic solvents (e.g., ethanol, propanol, DMSO), or application of solvent mixtures such as methanol and acetonitrile in various ratios.

In some embodiments, the disclosed method achieves a nucleic acid recovery of 50% or greater by area, based on the total area of the nucleic acid present in the LNP-encapsulated nucleic acid (i.e., in the sample prior to performing the disclosed method). In some embodiments, the disclosed method achieves a nucleic acid recovery of 80% or greater by area, based on the total area of nucleic acid present in the LNP-encapsulated nucleic acid.

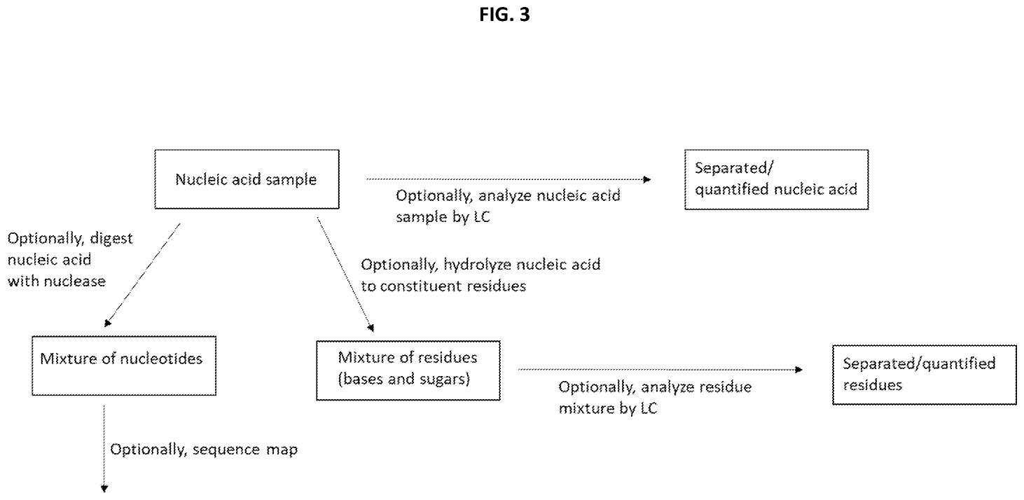

In some embodiments, the method further comprises analyzing the nucleic acid sample obtained by elution from the WAX sorbent. In some embodiments, the method further comprises processing the nucleic acid sample, and optionally, performing various analyses on the processed sample. A schematic illustration of some of these potential analyses and processing according to non-limiting embodiments is provided in FIG. 3.

In some embodiments, the method further comprises analyzing the nucleic acid sample, such as by a suitable liquid chromatographic method. Suitable liquid chromatographic methods include high performance and ultra-high performance liquid chromatography coupled with various detection methods including, but not limited to, tunable, dual wavelength ultraviolet/visible (TUV) detection, evaporative light scattering, mass spectrometry, charged aerosol, or refractive index.

In some embodiments, the method further comprises digesting with a nuclease at least a portion of the nucleic acid present in the nucleic acid sample. Digestion products include, but are not limited to, small (e.g., less than about 20 nucleotide) oligonucleotides, such as oligoribonucleotides. Examples of suitable nucleases include, but are not limited to, RNase T1 and Maz F. In some embodiments, the method further comprises analyzing the digested nucleic acid sample, such as by a suitable liquid chromatographic method. Suitable liquid chromatographic methods include high performance and ultra-high performance liquid chromatography coupled with various detection methods including, but not limited to, UV, evaporative light scattering, mass spectrometry, charged aerosol, and refractive index.

In some embodiments, the method further comprises sequence mapping of the digested nucleic acid mixture.

In some embodiments, the method further comprises hydrolyzing at least a portion of the nucleic acid present in the nucleic acid sample to form a mixture of constituent residues. In some embodiments, the method further comprises analyzing the constituent residues obtained by the hydrolysis (e.g., by performing liquid chromatography to separate and/or quantify the bases and/or sugars present in the mixture).

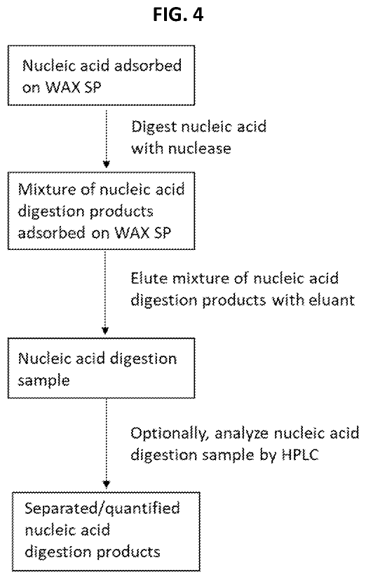

In other embodiments, the method further comprises digesting at least a portion of the solubilized nucleic acid adsorbed on the sorbent, forming an adsorbed mixture of nucleic acid digestion products, wherein digesting comprises contacting the sorbent with a nuclease; and eluting the mixture of nucleic acid digestion products from the WAX solid phase sorbent, forming a nucleic acid digestion sample. A flowchart depicting the method according to one non limiting embodiment of the disclosure is provided in FIG. 4. Each of the further method steps are described further herein below.

With continued reference to FIG. 4, in some embodiments, the adsorbed nucleic acid is subjected to digestion with a nuclease while remaining adsorbed on the sorbent. Surprisingly, it has been found according to the present disclosure that contacting the sorbent adsorbed nucleic acid with a nuclease results in digestion of at least a portion of the bound nucleic acid and the resulting digestion profile is comparable to that obtained during the digestion and analysis of the mRNA itself. This discovery makes it possible to quickly process the disrupted LNP mRNA without the need for further precipitation techniques. As described herein above, to disrupt an LNP, alcoholic solutions are generally needed, and this presents a problem for enzymatic digestion when performed in the absence of the described WAX separation method. Particularly, the components of the lipid nanoparticle are also dissolved into solution, complicating the analysis. Further, as demonstrated herein in Example 2, nuclease digestion of an mRNA sample failed when performed directly in the disruption solution.

In a non-limiting embodiment of the disclosed method, and with reference to FIGS. 1, 2 and 4, the LNP-nucleic acid formulation is disrupted, the mixture applied to a WAX sorbent, and the lipid components are removed, each as described herein above. However, in this particular embodiment, the adsorbed nucleic acid is digested while remaining adsorbed on the WAX sorbent. With continued reference to FIG. 4, following the digestion, the mixture of nucleic acid digestion products so obtained is eluted from the sorbent with an eluant, forming a nucleic acid digestion sample.

In some embodiments, eluting the mixture of nucleic acid digestion products comprises flowing an aqueous buffer through the WAX solid phase sorbent. The aqueous buffer utilized for elution of the adsorbed nucleic acid may vary but is generally an aqueous buffer having a pH greater than or equal to the pKa of the anion exchanger residues of the WAX solid phase sorbent. In some embodiments, the aqueous buffer has a pH in a range from about 8 to about 12.5, such as from about 8, or about 9, to about 10, about 11, or about 12. In some embodiments, the aqueous buffer has a pH in a range from about 8.5 to about 12, such as about 8.5, about 9, about 9.5, about 10, about 10.5, about 11, about 11.5, or about 12. Suitable buffers include, but are not limited to, aqueous ammonium bicarbonate, ammonium acetate, ammonium formate, triethylamine, tetramethylammonium, hexylamine, diisopropylethylamine, and ammonium hydroxide.

In some embodiments, the aqueous buffer further comprises a non-aqueous solvent. The non-aqueous solvent may be present in various ratios with the aqueous buffer, and such ratios may be determined experimentally by one of skill in the art. In some embodiments, the eluant comprises up to about 50% by volume of the non-aqueous solvent. In some embodiments, the non-aqueous solvent is an organic solvent as described herein. In some embodiments, the aqueous buffer further comprises a C1-C4 alcohol, acetonitrile, dimethyl formamide, dimethyl sulfoxide, or a combination thereof.

In some embodiments, the method further comprises analyzing the nucleic acid digestion sample, providing separated and/or quantified nucleic acid digestion products. Suitable methods for analyzing the sample include, but are not limited to, high performance liquid chromatography techniques such as UPLC, reversed-phase HPLC, ion-pairing HPLC, and the like, coupled with various detection methods including, but not limited to, tunable, dual wavelength ultraviolet/visible (TUV) detection, evaporative light scattering, mass spectrometry, charged aerosol, or refractive index.

All methods described herein can be performed in any suitable order unless otherwise indicated herein or otherwise clearly contradicted by context. The use of any and all examples, or exemplary language (e.g., “such as”) provided herein, is intended merely to better illuminate the materials and methods and does not pose a limitation on the scope unless otherwise claimed. No language in the specification should be construed as indicating any non-claimed element as essential to the practice of the disclosed materials and methods.

It will be readily apparent to one of ordinary skill in the relevant arts that suitable modifications and adaptations to the compositions, methods, and applications described herein can be made without departing from the scope of any embodiments or aspects thereof. The compositions and methods provided are exemplary and are not intended to limit the scope of the claimed embodiments. All of the various embodiments, aspects, and options disclosed herein can be combined in all variations. The scope of the compositions, formulations, methods, and processes described herein include all actual or potential combinations of embodiments, aspects, options, examples, and preferences herein.

Although the technology herein has been described with reference to particular embodiments, it is to be understood that these embodiments are merely illustrative of the principles and applications of the present technology. It will be apparent to those skilled in the art that various modifications and variations can be made to the method and apparatus of the present technology without departing from the spirit and scope of the technology. Thus, it is intended that the present technology include modifications and variations that are within the scope of the appended claims and their equivalents.

Reference throughout this specification to “one embodiment,” “certain embodiments,” “one or more embodiments” or “an embodiment” means that a particular feature, structure, material, or characteristic described in connection with the embodiment is included in at least one embodiment of the technology. Thus, the appearances of phrases such as “in one or more embodiments,” “in certain embodiments,” “in one embodiment” or “in an embodiment” in various places throughout this specification are not necessarily referring to the same embodiment of the technology. Furthermore, the particular features, structures, materials, or characteristics may be combined in any suitable manner in one or more embodiments. Any ranges cited herein are inclusive.

Aspects of the present technology are more fully illustrated with reference to the following examples. Before describing several exemplary embodiments of the technology, it is to be understood that the technology is not limited to the details of construction or process steps set forth in the following description. The technology is capable of other embodiments and of being practiced or being carried out in various ways. The following examples are set forth to illustrate certain aspects of the present technology and are not to be construed as limiting thereof.

### EXAMPLES

The present invention may be further illustrated by the following non-limiting examples describing the chromatographic devices and methods.

### Materials

All reagents were used as received unless otherwise noted. Those skilled in the art will recognize that equivalents of the following supplies and suppliers exist and, as such, any suppliers listed below are not to be construed as limiting.

### Example 1. Sample Preparation and Fractionation of Lipids and mRNA

A sample representative of a lipid nanoparticle (LNP) formulated mRNA was created from a mixture of lipid constituents and a nucleic acid. The nucleic acid was an erythropoietin (EPO) transcript based on 5-methoxy uridine modified mRNA (L-7209; available from TriLink Biotechnologies, San Diego, CA). An mRNA stock solution was prepared as a 100 μg/mL solution in 1 mM sodium citrate buffer (pH 6.4). The lipid component consisted of cholesterol (41.9%), an ionizable lipid constituent (ALC-0315; 45.4%), a pegylated lipid constituent (ALC-0159; 3.5%), and a phospholipid constituent (1,2-DSPC; 9.2%). The constituents were obtained from Cayman Chemical, Ann Arbor, MI (Lipid Nanoparticle Exploration Kit; Item number 35426). A lipid stock was created from this mixture, and contained 1.65 mg/mL cholesterol, 3.54 mg/mL of the ionizable lipid constituent, 0.78 mg/mL of the pegylated lipid constituent, and 0.74 mg/mL of the phospholipid. To form the sample mixture for fractionation, a portion of the lipid stock mixture (10 uL) and a portion of the mRNA stock solution (10 uL) were added to 80 uL of a solvent mixture (36% by volume of aqueous triethylammonium acetate; 100 mM; pH 10) and 64% by volume of n-propanol).

A well of an Oasis™ weak anion exchange (WAX) μElution Plate (available from Waters, Inc., Milford, MA) was conditioned for use by washing with 2% formic acid in methanol (2×200 μL) followed by 2% formic acid in water (2×200 μL). The sample mixture (100 μL) was applied to the well, and the lipids were collected in the pass-through fraction.

The lipid mixture was subsequently analyzed by reversed-phase high performance liquid chromatography (HPLC) using evaporative light scattering detection. The sample injection volume was 5 μL. The separation was performed using a commercially available HPLC system (ACQUITY™ UPLC™ H-Class Plus System, including and ACQUITY Photodiode Array Detector and an ACQUITY Evaporative Light Scattering Detector; all available from Waters Corporation, Milford, MA). The column was a Premier CSH Phenyl-Hexyl (1.7 μm 2.1×50 mm). The elution was performed at a temperature of 50° C. and a flow rate of 0.4 mL/minute with a linear gradient over 6 minutes of 100% mobile phase A to 100% mobile phase B, followed by a 2-minute hold at 100% mobile phase B. Mobile phase A was acetonitrile/isopropanol/0.1% aqueous formic acid water; 50/10/40 by volume). Mobile phase B was acetonitrile/isopropanol/0.1% aqueous formic acid; 80/10/10 by volume).

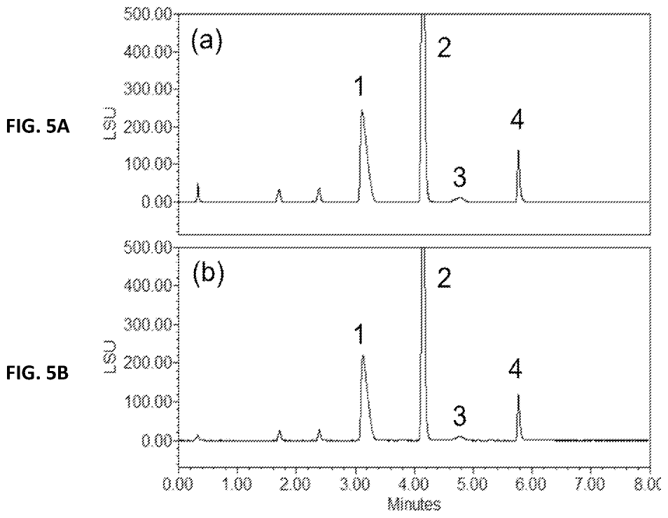

The results of the LC analysis of the lipid sample are provided in FIGS. 5A and 5B. FIG. 5A is a reference chromatogram of the mixture of lipids present in the lipid stock mixture (i.e., prior to performing the solid phase extraction procedure). FIG. 5B is a chromatogram of the lipid sample obtained as the pass-through fraction. For both FIGS. 5A and 5B, peak intensities of each lipid component present are provided as follows: Peak #1 is the ionizable lipid; peak #2 is cholesterol; peak #3 is the PEGylated lipid; and peak #4 is the phospholipid. Comparing FIGS. 5A and 5B, it can be observed that each of the lipids initially present in the sample were recovered in the pass-through fraction, and recovery was 84%. Further, the relative abundance of each lipid was unchanged by the extraction procedure.

The WAX elution plate cell containing the adsorbed mRNA was then processed to provide the mRNA component. Specifically, the elution plate was washed with methanol (2×50 μL), 2% formic acid in water (2×50 μL), and methanol (2×50 μL) to ensure complete removal of the lipid components. Then, the mRNA was eluted with aqueous ammonium bicarbonate (100 mM; pH 11) as the eluent (2×50 μL), followed by elution with methanol (2×50 μL).

The eluates were separately analyzed by ion pairing reversed phase high performance liquid chromatography with UV detection at 260 nm. The sample injection volume was 10 μL. The separation was performed using a commercially available HPLC system (ACQUITY™ UPLC™ H-Class Bio System, configured with an ACQUITY Photodiode Array Detector; each available from Waters Corporation, Milford, MA). The column was a Premier BEH C18 (1.7 μm, 2.1×50 mm). The elution was performed at a temperature of 50° C. and a flow rate of 0.4 mL/minute with a linear gradient over 3 minutes of 90% mobile phase A to 55% mobile phase B, followed by a 1-minute hold at 55% mobile phase B. Mobile phase A was 5/90/5 by volume of acetonitrile/water/1M triethylammonium acetate (pH 6.9). Mobile phase B was 60/35/5 by volume of acetonitrile/water/1M triethylammonium acetate (pH 6.9).

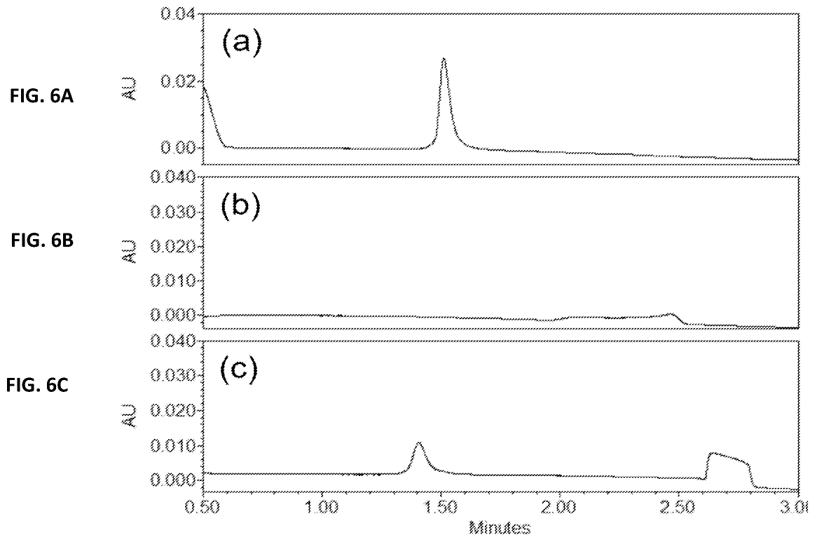

The results of the LC analysis of the mRNA sample are provided in FIGS. 6A-6C. FIG. 6A is a reference chromatogram of the mRNA present in the mRNA stock solution (i.e., prior to performing the solid phase extraction procedure). FIG. 6C is a chromatogram of the mRNA sample eluted from the WAX elution plate cell. With reference to FIG. 6C, 53% of the mRNA was recovered in the elute fractions (45.3% in the aqueous buffer eluant, and 7.4% in the methanol eluant; data not shown). FIG. 6B is a chromatogram of the lipid pass-through sample, showing that the mRNA was completely adsorbed by the WAX sorbent as evidenced by the clean baseline.

### Example 2. Attempted Nuclease Digestion of a Lipid Nanoparticle (LNP) Formulated mRNA in Disruption Solution

Messenger ribonucleic acid (mRNA; firefly luciferase transcript) formulated within a lipid nanoparticle (LNP-mRNA; 100 micrograms) was contacted with a disruption solution (an amine-containing alcohol solution, specifically, 35.7% of 0.1 mM triethylammonium acetate pH 10 buffer in n-propanol), followed by addition of endonuclease (RNaseT1; excess, ˜30 kU for 10 μg mRNA). The resulting mixture was analyzed by ion pairing reversed phase liquid chromatography and Mass Spectrometry (IP-RPLC-MS). The separation conditions were as follows:

### LC:

System: ACQUITY™ UPLC™ Premier System (available from Waters Corporation, Milford, MA)
Column: ACQUITY™ Premier Peptide BEH™ C18, 2.1×150 mm, 300 Å, 1.7 μm
Column Temperature: 70.0° C.
Solvent A: 1% 1,1,1,3,3,3-hexafluoroisopropanol (HFIP) and 0.1% N,N-diisopropylethylamine (DIPEA) in water
Solvent B: 0.075% HFIP/0.0375% DIPEA in 65% methanol 35% water

### MS:

Instrument: Vion IMS QTof
Instrument driver version: 4.0.0
Negative Mode ESI
MS^{E}, Mass range 50-4000 m/z, Scan time: 0.5 sec
Low collision energy: 6 V, High collision energy ramp: 10V-30V
Desolvation temperature: 600° C., Source temperature: 150° C.
Lock correction: Single reference mass at 554.2615 m/z

The separation was performed using gradient elution according to Table 1.

TABLE 1
Gradient elution conditions

| Time | Flow Rate | Solvent A | Solvent B | |
|---|---|---|---|---|
| (min) | (mL/min) | (%) | (%) | Curve |
| 0.00 | 0.300 | 97.0 | 3.0 | Initial |
| 25.00 | 0.300 | 45.0 | 55.0 | 6 |
| 27.00 | 0.300 | 5.0 | 95.0 | 6 |
| 27.10 | 0.300 | 5.0 | 95.0 | 6 |
| 30.00 | 0.300 | 97.0 | 3.0 | 6 |

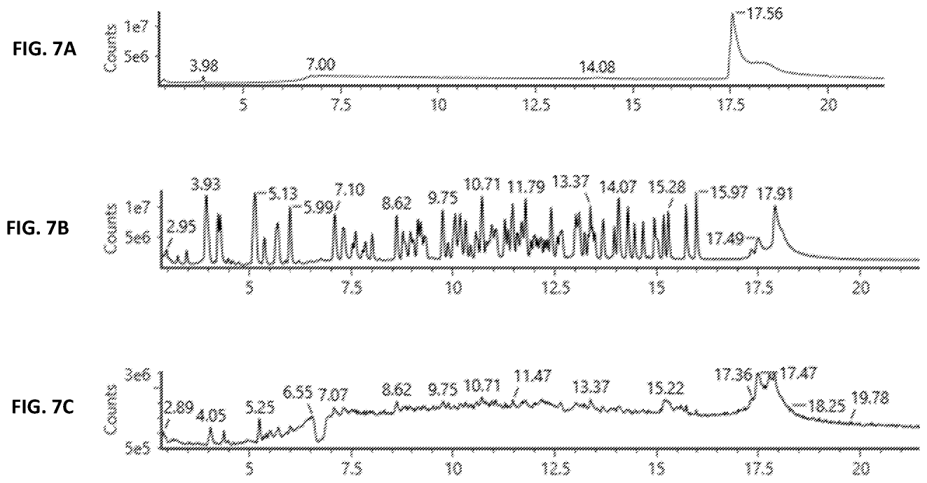

An exemplary reference chromatogram from an injection of undigested (intact) mRNA (firefly luciferase; non-lipid encapsulated) is shown in FIG. 7A. An exemplary reference chromatogram from an injection of positive control (standard, in-solution RNase T1 digestion of the same mRNA is shown as FIG. 7B. With reference to FIG. 7B, peaks corresponding to the separated mRNA digestion products are clearly visible, and the peak at retention time of about 17.5 minutes (corresponding to the intact mRNA) is greatly reduced in intensity. A chromatogram from the disruption/digestion solution is provided as FIG. 7C. With reference to FIG. 7C, while some digestion may have occurred, it is observed that no significant signal for digestion components was obtained.

### Example 3. On-Sorbent Nuclease Digestion of a Lipid Nanoparticle (LNP) Formulated mRNA

Messenger ribonucleic acid (mRNA; firefly luciferase transcript) formulated within a lipid nanoparticle (LNP-mRNA) was digested on a solid phase extraction (SPE) anion exchange sorbent. The anion exchange sorbent used was 20 μm calcined silica with a 300 Å pore and a 1.4 μmol/m² N,N-Bis [3-(methylamino)propyl] methylamine silane bonding (Daisogel USA, Torrance, CA). The digestion was performed in a 96-well plate format, with each well packed with a 4 mg bed of the sorbent. The sorbent was conditioned with methanol followed by 2% formic acid in water.

The LNP-mRNA formulation (˜100 μg) was disrupted with an amine-containing alcohol solution (triethylammonium acetate and n-propanol). The disrupted formulation was introduced to the well containing the sorbent material followed by addition of endonuclease (RNaseT1; excess, ˜30 kU for 10 μg mRNA). The sorbent plate was covered and placed in an oven at 37° C. for 30 minutes. Digest components were eluted from the sorbent using 40 μl of 10 mM Tris, 0.1 mM EDTA, pH 7.5, followed by elution with 40 μl of pH 10 disruption buffer. Vacuum was applied between the two elutions. The eluates were concentrated (˜10×) and reconstituted in 10 mM Tris, 0.1 mM EDTA, pH 7.5. The reconstituted sample was analyzed by IP-RPLC-MS as described in Example 2.

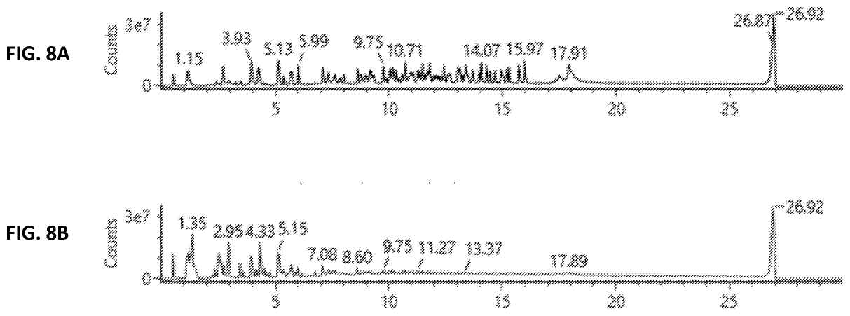

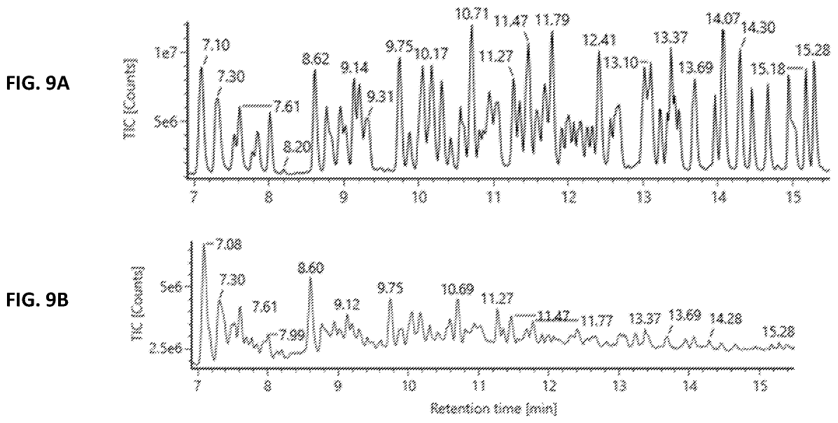

An exemplary reference chromatogram from an injection of positive control (standard, in-solution RNase T1 digestion of the same mRNA) is shown in FIG. 8A, and an expanded view relative to FIG. 8A is provided as FIG. 9A. With reference to FIGS. 8A and 9A, peaks corresponding to the separated mRNA digestion products are clearly visible, and the peak at retention time of about 17.9 minutes (corresponding to the intact mRNA) is greatly reduced in intensity. A chromatogram from the on-sorbent nuclease digestion sample is provided as FIG. 8B and an expanded view relative to FIG. 8B is provided as FIG. 9B. With reference to FIGS. 8B and 9B, it is observed that the mRNA within the LNP-mRNA formulation was digested on sorbent. The intensity of digest components eluted following the on-sorbent digestion was lower than in the case of free mRNA digestion (about 1 order of magnitude), but the chromatographic features were highly reproducible (FIGS. 8B and 9B). These results also demonstrate that the on-sorbent digestion profile of mRNA from an LNP-mRNA formulation is comparable to the digestion profile of unformulated mRNA (i.e., the method is digesting the mRNA portion of LNP-mRNA formulation, producing the same distribution of digestion products).

What is claimed is:

1. A method of preparing a sample for compositional analysis, the method comprising:
providing a sample comprising a lipid particle encapsulated nucleic acid;
dissolving the sample comprising the lipid particle encapsulated nucleic acid in an organic solvent to provide a solution comprising dissolved lipids and solubilized nucleic acid; and
adsorbing the solubilized nucleic acid on a weak anion exchange (WAX) solid phase sorbent.

2. The method of claim 1, wherein the WAX solid phase sorbent comprises a surface having anion exchanger residues disposed thereon, the anion exchanger residues having a pKa in a range from about 5 to about 11, the anion exchanger residues comprising a primary amine, a secondary amine, a tertiary amine, or a combination thereof.

3. The method of claim 1, wherein the anion exchanger residues comprise piperazine.

4. The method of claim 1, wherein the organic solvent comprises a C1-C4 alcohol, acetonitrile, dimethyl formamide, dimethyl sulfoxide, or a combination thereof.

5. The method of claim 1, wherein the organic solvent comprises n-propanol.

6. The method of claim 1, wherein the organic solvent further comprises an additive selected from the group consisting of acids, bases, detergents, buffers, salts, and combinations thereof.

7. The method of claim 6, wherein the additive comprises an ionizable cationic base.

8. The method of claim 1, further comprising removing the dissolved lipids from the WAX solid phase sorbent, forming a lipid sample which is substantially free of nucleic acid, wherein removing the dissolved lipids comprises allowing the dissolved lipids to pass through the WAX solid phase sorbent.

9. The method of claim 8, wherein removing the dissolved lipids further comprises:
flowing the organic solvent through the WAX solid phase sorbent and collecting the organic solvent flowed therethrough to provide one or more washings; and
combining the one or more washings with the dissolved lipids which have been allowed to pass through the WAX solid phase sorbent.

10. The method of claim 1, further comprising eluting the adsorbed nucleic acid from the WAX solid phase sorbent, forming a nucleic acid sample which is substantially free of lipids.

11. The method of claim 10, wherein eluting comprises flowing an aqueous buffer through the WAX solid phase sorbent, wherein the aqueous buffer has a pH greater than or equal to the pKa of the anion exchanger residues of the WAX solid phase sorbent, said aqueous buffer comprising up to about 50% by volume of a non-aqueous solvent.

12. The method of claim 11, wherein the aqueous buffer has a pH from about 8 to about 12.5, or from about 8.5 to about 12.

13. The method of claim 1, further comprising analyzing one or more of the lipid sample and the nucleic acid sample by high performance liquid chromatography, mass spectrometry, or a combination thereof.

14. The method of claim 10, further comprising digesting at least a portion of the nucleic acid present in the nucleic acid sample with a nuclease.

15. The method of claim 10, further comprising hydrolyzing at least a portion of the nucleic acid present in the nucleic acid sample to form a mixture of constituent residues.

16. The method of claim 1, further comprising:
digesting at least a portion of the solubilized nucleic acid adsorbed on the sorbent, forming an adsorbed mixture of nucleic acid digestion products, wherein digesting comprises contacting the sorbent with a nuclease; and
eluting the mixture of nucleic acid digestion products from the WAX solid phase sorbent, forming a nucleic acid digestion sample.

17. The method of claim 16, wherein eluting comprises flowing an aqueous buffer through the WAX solid phase sorbent, wherein the aqueous buffer has a pH greater than or equal to the pKa of the anion exchanger residues of the WAX solid phase sorbent, and wherein said aqueous buffer comprises up to about 50% by volume of a non-aqueous solvent.

18. The method of claim 11, wherein the non-aqueous solvent comprises a C1-C4 alcohol, acetonitrile, dimethyl formamide, dimethyl sulfoxide, or a combination thereof.

19. The method of claim 17, wherein the aqueous buffer has a pH of about 7 to about 12.5, or from about 8 to about 12.

20. The method of claim 16, further comprising analyzing the nucleic acid digestion sample by high performance liquid chromatography, mass spectrometry, or a combination thereof.
